(19) United States
(12) Patent Application Publication
(10) Pub. No.: US 2024/0371503 A1
(43) Pub. Date: Nov. 7, 2024

(54) SYSTEMS AND METHODS FOR EVALUATING HOSPITAL CARE OF INFECTION PATIENTS USING LENGTH-OF-STAY ESTIMATION MODELS

(71) Applicant: KONINKLIJKE PHILIPS N.V., EINDHOVEN (NL)

(72) Inventors: Ting Feng, Cambridge, MA (US); Robert Damiano, Cambridge, MA (US); Seyed Morteza Miran, Cambridge, MA (US); Arad Lajevardi-Khosh, Cambridge, MA (US)

(21) Appl. No.: 18/654,666

(22) Filed: May 3, 2024

Related U.S. Application Data
(60) Provisional application No. 63/463,983, filed on May 4, 2023.

(51) Int. Cl. G16H 40/20; G16H 10/60; G16H 50/30

(52) U.S. Cl. CPC G16H 40/20; G16H 10/60; G16H 50/30

(57) ABSTRACT

Systems and methods for infection treatment evaluation is provided. The system receives patient encounter records including patient attribute data and patient LoS data. The system generates, via an LoS estimation model, LoS estimations based on the patient attribute data of each of the patient encounter records. The patient attribute data includes vital sign data, patient demographic data, laboratory data, medical condition data, infection type data, medication administration data, and/or discharge type. The system further generates, via a metric analyzer, evaluation metrics based on the LoS estimations and the patient LoS data of each of the patient encounter records. The system may also include a user interface configured to display at least a portion of the one or more evaluation metrics. The system may also train the LoS estimation model with a historical encounter records. Each of the historical encounter records includes historical attribute data and historical recovery data.

### CROSS REFERENCE TO RELATED APPLICATION

The present application is a continuation of co-pending U.S. Patent Application Ser. No. 63/463,983, filed May 4, 2023. This application is hereby incorporated by reference herein.

### FIELD OF THE DISCLOSURE

The present disclosure is generally directed to operational intelligence solutions in the healthcare domain and, more particularly, to systems and methods for evaluating hospital care of infection patients.

### BACKGROUND

When patient transition decisions are made at both enterprise and unit level at a hospital or other healthcare facility, priorities and bottlenecks may be difficult to identify-potentially leading to inconsistent care delivery. To better orchestrate and manage patient care, it is essential to combine clinical and operational data, translating it into actionable information, helping healthcare professionals manage their patients across the care spectrum.

A common problem for a hospital operations team is evaluating how well the hospital manages patient flow, particularly for patients admitted due to infectious disease, or for patients being admitted for a different reason, but being subsequently treated for a hospital-acquired infection. This is a challenging task because the initial host response to infectious agent and recovery trajectory can vary significantly by individual, by subgroup of patients, by the type of infectious agent, and by the main body system or organ that the infectious agent targets.

### SUMMARY OF THE DISCLOSURE

The present disclosure relates to a cloud-based patient logistics management solution, and, more particularly, and is directed to systems and methods for evaluating hospital care of infection patients. The systems and methods according to various embodiments and implementations of the solution disclosed herein employ a length of stay (LoS) estimation model to estimate the recovery time of a plurality of infection patients based on attributes of each patient. The LoS estimation model is a machine learning model trained by historical patient encounter records. One or more evaluation metrics are generated by comparing the estimated recovery time of each patient to their actual length of stay.

The LoS estimation model is configured to estimate the time of recovery for in-patient infection treatment of an individual patient. Time of recovery is defined as the time from hospital admission to routine discharge to home. The LoS estimation model utilizes a time-to-event prediction (also known as survival analysis) algorithm to generate an LoS estimation based on attributes of the patient. Further, the LoS estimation model is specifically configured to generate LoS estimates for patients meeting three criteria: (1) patients diagnosed as infection patients according to an International Classification of Disease (ICD) code associated with the patient; (2) patients who are adults (meaning patients eighteen years old and above); and (3) patients receiving inpatient, rather than outpatient, treatment.

The attributes used by the LoS estimation model to generate an LoS estimate may include vital sign data (such as heart rate, blood pressure, temperature, etc.), patient demographic data (such as age, gender, body mass index, etc.), laboratory data (such as metabolic panels, complete blood count, arterial blood gas, etc.), underlying medical condition data (high blood pressure, diabetes, etc.), infection type data (respiratory, genitourinary, skin and soft tissue, gastrointestinal, circulatory, central nervous system, or cardiovascular), and medication administration data (type of medication, frequency of medication, dosage of medication). The vital sign data and the laboratory data both correspond, for example, to the first twelve hours of inpatient stay. Further, the system may be configured to generate comorbidity index data based on aspects of the aforementioned patient attributes. The comorbidity index may also be used by the LoS estimation model to generate the LoS estimate. The patient attributes may be extracted from electronic medical records (EMR).

The LoS estimation model is trained using historical patient encounter records. Each historical patient encounter record corresponds to a previous patient. In some examples, the historical patient encounter records include patients treated at many different healthcare facilities over a period of many years. The historical patient encounter records include historical patient attribute data and historical recovery data. The historical patient encounter records may be extracted from historical EMR.

The historical patient attribute data may include many of the same types of attributes as listed above, including vital sign data, patient demographic data, laboratory data, underlying medical condition data, infection type data, and medication administration data. The historical patient attribute data may also include discharge type (routine discharge to home, discharged for further treatment, or discharged to hospice care/deceased) and comorbidity index.

The historical recovery data represents the amount of time from hospital admission to recovery, typically corresponding to the time of discharge. The historical recovery data may be right censored according to the discharge type of the patient attribute data. The right censored recovery data is a subset of the recovery data where the patient was discharged prior to recovery. In particular, historical recovery data corresponding to patients who were discharged for further treatment, discharged to hospice care, or deceased, is labeled as right censored, as the patient has not yet recovered as of the time of discharge.

The LoS estimation model may include several adjustable regularization parameters. The regularization parameters may be adjusted to prevent overfitting to training data (in this case, the historical patient encounter records) during the training process. The regularization parameters are adjusted based on a concordance index value of the LoS estimation model. The concordance index is determined based on five-fold cross-validation of the LoS estimation model. Each of the five folds represents an equal portion of the historical patient encounter records used to train the LoS estimation model. Further, the historical patient encounter records are stratified across the folds according to gender and discharge type. Thus, each fold has an equal male-to-female ratio as well as an equal ratio of the three discharge types (routine discharge to home, discharged for further treatment, or discharged to hospice care/deceased).

The system further generates metrics evaluating hospital performance by comparing LoS estimations to the actual LoS for each patient of a test group. For example, evaluation metrics may be generated for every infection patient admitted into a hospital during a one-month period. The evaluation metrics show if infection patients during that month had a slower or faster recovery time than estimated. These recovery times can be broken down by a number of factors, such as any of the aforementioned patient attributes or total infection patient admittance.

Generally, in one aspect, a system for infection treatment evaluation is provided. The system includes a controller. The controller has a processor and a memory. The controller is configured to receive one or more patient encounter records. Each of the one or more patient encounter records includes patient attribute data and patient LoS data.

The controller is further configured to generate, via an LoS estimation model, one or more LoS estimations. The one or more LOS estimations are generated based on the patient attribute data of each of the one or more patient encounter records. According to an example, the patient attribute data includes vital sign data, patient demographic data, laboratory data, medical condition data, infection type data, medication administration data, and/or discharge type.

The controller is further configured to generate, via a metric analyzer, one or more evaluation metrics. The one or more evaluation metrics are generated based on the one or more LoS estimations and the patient LoS data of each of the one or more patient encounter records.

According to an example, the system further comprises a user interface. The user interface is configured to display at least a portion of the one or more evaluation metrics.

According to an example, the controller is further configured to extract, via a feature extractor, at least a portion of the one or more patient encounter records from contemporary EMR.

According to an example, the controller is further configured to train the LoS estimation model with a plurality of historical encounter records. Each of the plurality of historical encounter records includes historical attribute data and historical recovery data. In one example, the historical recovery data includes right-censored recovery data. The right-censored recovery data corresponds to patient discharge prior to recovery. In another example, the historical attribute data comprises historical vital sign data, historical demographic data, historical laboratory data, historical medical condition data, historical infection type data, historical medication administration data, and/or historical discharge type.

According to an example, the controller is further configured to extract, via a feature extractor, at least a portion of the plurality of historical encounter records from historical EMR.

According to an example, the controller is further configured to generate, via the plurality of historical encounter records, a concordance index value of the LoS estimation model. In some examples, the controller is further configured to divide the plurality of historical encounter records into five folds. The plurality of historical encounter records may be divided in the five folds based on historical gender and historical discharge type. The controller may be further configured to adjust one or more regularization parameters of the LoS estimation model. The regularization parameters are adjusted based on the concordance index value.

Generally, in another aspect, a method for infection treatment evaluation is provided. The method includes receiving, via a controller having a processor and a memory, one or more patient encounter records, wherein each one of the one or more patient encounter records comprises patient attribute data and patient LoS data.

The method further includes generating, via an LoS estimation model of the controller, one or more LOS estimations based on the patient attribute data of each of the one or more patient encounter records.

The method further includes generating, via a metric analyzer of the controller, one or more evaluation metrics based on the one or more LOS estimations and the patient LoS data of each of the one or more patient encounter records.

According to an example, the method further includes displaying, via a user interface, at least a portion of the one or more evaluation metrics.

According to an example, the method further includes training the LoS estimation model with a plurality of historical encounter records, wherein each of the plurality of historical encounter records comprises historical attribute data and historical recovery data.

In various implementations, a processor or controller may be associated with one or more storage media (generically referred to herein as “memory,” e.g., volatile and non-volatile computer memory such as RAM, PROM, EPROM, EEPROM, floppy disks, compact disks, optical disks, magnetic tape, SSD, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a functional block diagram of a system for infection treatment evaluation, in accordance with an example.

FIG. 2 is another functional block diagram of a system for infection treatment evaluation, in accordance with an example.

FIG. 3 is a user interface of a system for infection treatment evaluation, in accordance with an example.

FIG. 4 is a further embodiment of a user interface of a system for infection treatment evaluation, in accordance with an example.

FIG. 5 is a further embodiment of a user interface of a system for infection treatment evaluation, in accordance with an example.

FIG. 6 is a schematic diagram of a controller of a system for infection treatment evaluation, in accordance with an example.

FIG. 7 is a schematic diagram of aspects of a memory of the controller of FIG. 6, in accordance with an example.

FIG. 8 is a flow chart of a method for infection treatment evaluation, in accordance with an example.

### DETAILED DESCRIPTION OF EMBODIMENTS

The present disclosure is directed to systems and methods for evaluating hospital care of infection patients. The systems and methods employ a length of stay (LoS) estimation model to estimate the recovery time of a plurality of infection patients based on attributes of each patient. The LoS estimation model is a machine learning model trained by historical patient encounter records. One or more evaluation metrics are generated by comparing the estimated recovery time of each patient to their actual length of stay.

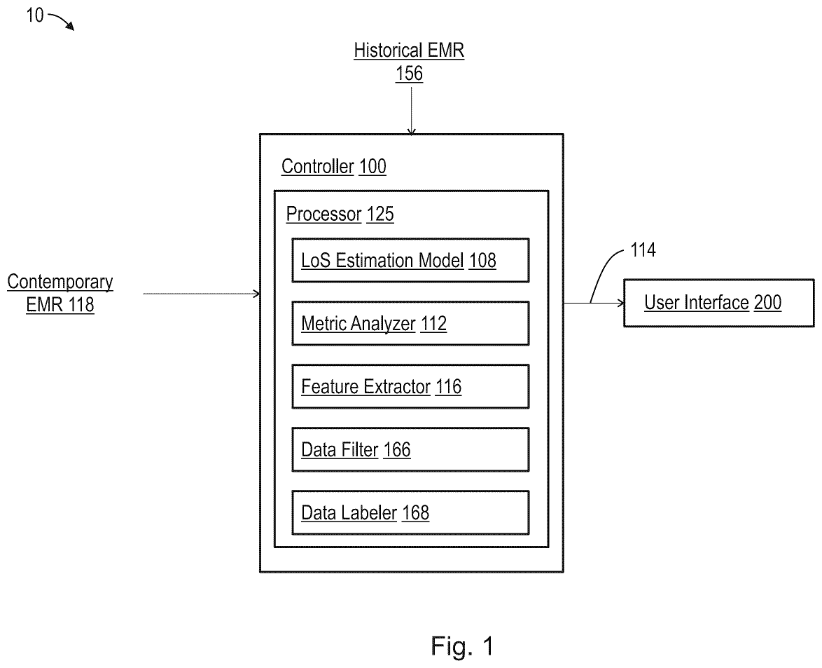

FIG. 1 illustrates a system 10 for infection treatment evaluation in a hospital or other healthcare facility. Generally, the system 10 evaluates the treatment progression of infection patients by comparing an individualized expected LoS of to their actual LoS. In this system 10, a longer than expected LoS is generally indicative of a low quality of care. In some examples, a longer than expected LoS may be a result of a failure to properly treat the infection of the patient, or the result of a re-infection of a different type of infection contracted in the hospital or healthcare facility. By evaluating the treatment progression of infection patients, the hospital or healthcare facility can adjust its own internal practices and procedures to improve the quality of care and decrease patient LoS.

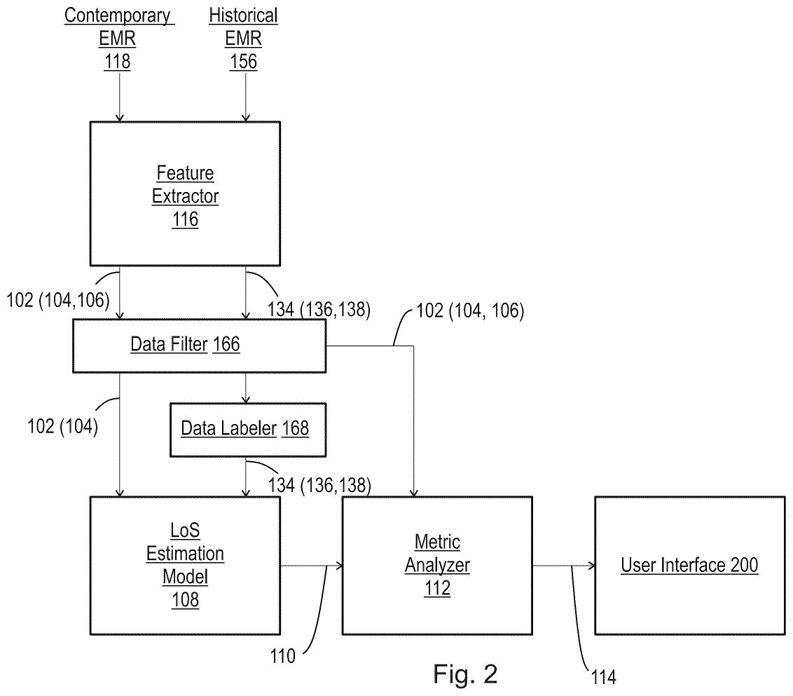

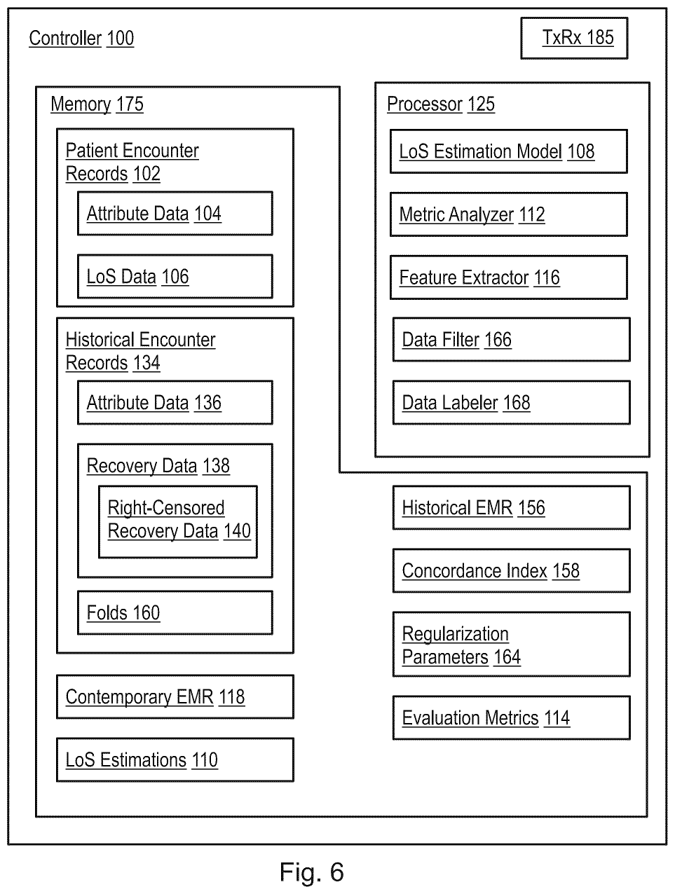

Broadly, the system 10 includes a controller 100. The controller 100, shown in more detail in FIG. 6, includes a processor 125, a memory 175, and a transceiver 185. The processor 125 is configured to execute a variety of models to estimate or predict various aspects of the system 10. In the example of FIG. 2, the processor 125 is configured to execute an LOS estimation model 108, a metric analyzer 112, a feature extractor 116, a data filter 166, and a data labeler 168. These models are described in further detail with reference to FIG. 2. The memory 175 is configured to store data received by the controller 100 via wired or wireless connection. The memory 175 is further configured to store data generated by the processor 125.

The transceiver 185 is configured to wirelessly transmit data to or wirelessly receive data from various aspects of the system 10. The transceiver 185 may also enable wireless communication with components arranged externally to the system 10. In the example of FIG. 2, the transceiver 185 may wirelessly receive contemporary electronic medical records (EMR) 118. The contemporary EMR 118 includes medical records of infection patients who have been recently treated in the hospital or care facility. In some examples, the contemporary EMR 118 includes records of patients who have been admitted, treated, and discharged to home within the last thirty days. As will be explained in more detail with respect to FIG. 2, the system 10 extracts and processes information from the contemporary EMR 118 to evaluate the quality of treatment in the hospital or healthcare facility.

The transceiver 185 may also wirelessly receive a wide array of training data. This training data is used to train machine learning or artificial intelligence models executed by the processor 125. For example, the transceiver 185 may enable the reception of historical EMR 156, which is subsequently processed and used to train the LoS estimation model 108.

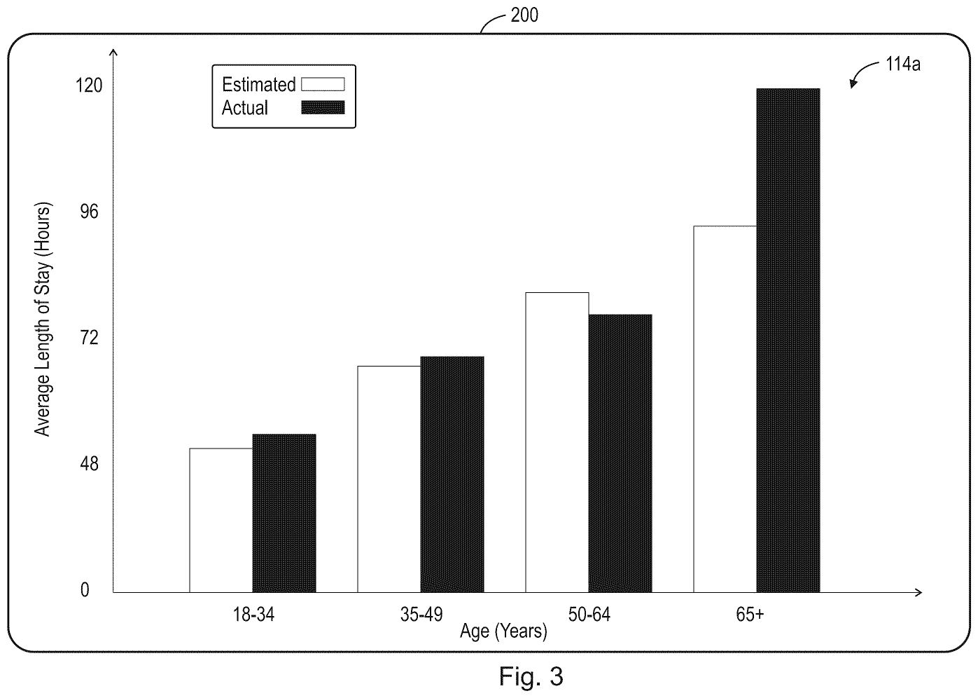

The system 10 also includes a user interface 200. Non-limiting examples of the user interface 200 are shown in FIGS. 3-5. In some examples, the user interface 200 may include a display screen to show evaluation metrics 114 and/or other data generated by the controller 100. The evaluation metrics 114 may be conveyed to the user interface 200 via wired or wireless connection, such as via the transceiver 185 of the controller 100.

FIG. 2 is another functional block diagram of a system 10 for infection treatment evaluation. In particular, FIG. 2 illustrates the flow of data between the various models and programs executed by the processor 125 of the controller 100 to generate metrics evaluating the treatment of infection patients in a hospital or other type of healthcare facility.

As shown in FIG. 3, the system includes an LOS estimation model 108. The LoS estimation model 108 is a predictive model configured to estimate the length of time an infection patient will stay in the hospital or healthcare facility. This length of stay is also referred to as the “recovery time,” as the discharge of the patient generally corresponds with the patient recovering from the infection. The LoS estimation model 108 typically utilizes a time-to-event algorithm (also known as survival analysis).

The LoS estimation model 108 receives patient encounter records 102. Each patient encounter record 102 corresponds to an individual infection patient being admitted to the hospital or healthcare facility under evaluation. Each patient encounter record includes patient attribute data 104 and patient LoS data 106. The patient attribute data 104 includes a wide array of data related to the characteristics of the patient. In particular, at least a portion of these patient attributes are considered to be useful datapoints in estimating the LoS of the patient. The LoS data 106 indicates the amount of time from admission of the patient to the hospital or healthcare facility to routine discharge.

As shown in FIG. 2, the patient encounter records 102 may be extracted from contemporary EMR 118 via a feature extractor 116. The feature extractor 116 may process the contemporary EMR 118 using any appropriate means.

The patient encounter records 102 may include a wide array of data, including vital sign data 120, patient demographic data 122, laboratory data 124, medical condition data 126, infection type data 128, medication administration data 130, and/or discharge type 132. The vital sign data 120 may include measurements such as heart rate, blood pressure, temperature, etc. The patient demographic data 122 may include information such as age, gender, body mass index (BMI), etc. The laboratory data 124 may include measurements (or collections of measurements) such as metabolic panels, complete blood count, and arterial blood gas data. The medical condition data 126 provides information regarding any underlying medical conditions the patient may have, such as high blood pressure, diabetes, etc. The infection type data 128 indicates the type of infection contracted by the patient, such as respiratory, genitourinary, skin and soft tissue, gastrointestinal, circulatory, central nervous system, or cardiovascular. The medication administration data 130 includes information regarding medication the patient is currently taking, such as type of medication, frequency of medication, and/or dosage of medication. This medication may or may not be related to the current infection of the patient.

The vital sign data 120 and the laboratory data 124 include measurements taken within the first twelve hours of admission to the healthcare facility. In addition to the raw measurements (such as a heart rate of 80 beats per minute), the vital sign data 120 and the laboratory data 124 may include statistical values based on the measurements, including median values, mean values, max values, standard deviation values, and range values.

In some examples, the patient attribute data 104 may include additional information. For example, the patient attribute data 104 may include a discharge type 132 for the patient if they have been discharged. The discharge type 132 indicates whether the patient was routinely discharged to home or another non-care facility, discharged for further treatment (such as to a skilled nursing facility, acute rehabilitation, long term care hospital, or home care with home health services), or discharged to hospice care or otherwise deceased.

The patient attribute data 104 may also include a disease code, such as a code as defined by the International Classification of Diseases (ICD). The code may be defined under the ICD-9, ICD-10, or ICD-11 standards. The patient attribute data 104 may also include a comorbidity index, such as the Elixhauser comorbidity index (ECI). The ECI value for a patient may be determined based on diagnostic ICD codes and a set of thirty comorbidity categories. In some examples, an aspect of the system 10 derives the ECI value from other values of the patient attribute data 104. For example, the ECI value could be generated based in part on demographic data 122 and medical condition data 126.

The patient encounter records 102 may be filtered via a data filter 166 prior to receipt by the LoS estimation model 108. Generally, the data filter 166 may filter the patient encounter records 102 to form a cohort which may be predictably processed by the LoS estimation model 108. First, the filter 166 limits the patient encounter records 102 to patients having been diagnosed with an infection within three hours of admission. Accordingly, patients who have been diagnosed with other ailments, such as trauma, burns, and/or cardiac issues are excluded. This filtering may be based on ICD codes included in the patient attribute data 104.

The patient encounter records 102 may be further limited to patients eighteen years of age or older. This filtering may be based on the demographic data 122 of the patient attribute data 104.

The patient encounter records 102 may be further limited to patients receiving inpatient treatments. Inpatient treatments correspond to lengths of stay of 24 hours or more. This filtering may be based on patient LoS data 106.

Once the patient encounter records 102 are filtered, the LoS estimation model 108 generates an LoS estimation 110 for each patient. The LoS estimations 110 indicate the expected length of stay for each patient based on the information in their patient encounter record 102. These estimates 110 are used to evaluate the quality of treatment of the hospital or healthcare facility.

However, in order to generate the LoS estimations 110, the LoS estimation model 108 is trained with historical encounter records 134. Similar to the patient encounter records 102 used to estimate a length of stay, the historical encounter records 134 represent past patient encounters for infection treatment. Optimally, the historical encounter records 134 reflect a wide array of patient encounters across many different hospitals and healthcare facilities over a period of many years. The historical records 134 include historical attribute data 136 and historical recovery data 138. Like the aforementioned patient attribute data 104, the historical attribute data 136 may include a wide array of information indicative of recovery time such as historical vital sign data 142, historical patient demographic data 144, historical laboratory data 146, historical medical condition data 148, historical infection type data 150, historical medication administration data 152, and/or historical discharge type 154.

Like the patient encounter records 102, the historical encounter records 134 may be extracted from historical EMR 156 via the feature extractor 116. The feature extractor 116 may process the historical EMR 156 using any appropriate means.

Further, prior to using the historical encounter records 134 to train the LoS estimation model 108, the historical encounter records 134 are filtered and labeled. First, like the patient encounter records 102, data filter 166 limits the historical encounter records 134 to adult, inpatient (length of stay of at least 24 hours) infection patients. Second, a data labeler 168 is used to right-censor certain historical encounter records 134 where the true recovery time of the historical patient is unknown. In particular, historical encounter records 134 where the patient is not routinely discharged to home, but instead discharged for further treatment (such as to a skilled nurse facility, acute rehabilitation, long term care hospital, or home care with home health services), or discharged to hospice care or otherwise deceased, are right censored, as the patient had not fully recovered upon discharge, transfer, or death. Accordingly, the discharge type 154 of the historical encounter records 134 determines whether or not the record is right censored by the data labeler 168.

Once filtered and labeled, the historical encounter records 134 are used to train the LoS estimation model 108. In one example, the LoS estimation model 108 is trained using five-fold cross validation to prevent overfitting. Accordingly, during training, at least a portion of the historical patient encounters 134 is divided into five folds 160. Each fold of the five folds 160 includes an equal number of historical patient encounters 134. Further, the historical patient encounters 134 are stratified across the five folds 160 by gender 162 and discharge type 154. Accordingly, each of the five folds 160 includes an equal number of male-to-female ratio. Further, the different varieties of discharge type 154 are evenly distributed across the five folds 160. While the aforementioned example uses five folds 160 for cross-validation, more than or less than five folds may be used in other examples.

During cross-validation, a concordance index value 158 for the trained LoS estimation model 108 is calculated. One or more regularization parameters of the 164 of the LoS estimation model 108 may be adjusted to optimize the concordance index value 158.

In some examples, several different LoS estimation models 108 may be trained, each LoS estimation model trained with a different subset of the filtered and labeled historical encounter data 134. In one example, a first LoS estimation model 108a is trained solely with historical attribute data 136 consisting of only historical vital sign data 142 and historical demographic data 144. In another example, a second LoS estimation model 108b is trained with historical attribute data 136 consisting of historical vital sign data 142, historical demographic data 144, and historical laboratory data 146. In a further example, a third LoS estimation model 108c is trained with historical attribute data 136 consisting of historical vital sign data 142, historical demographic data 144, historical laboratory data 146, and historical infection type data 150. Once each of the LoS estimation models 108a-c trained, the models 108a-c can be evaluated (such as by concordance index value 158a-c) to choose one of the models 108a-c. While the LoS estimation model 108 trained with the most data will typically be the most accurate, in some cases a model requiring less data that is only slightly less accurate may be preferred. For example, contemporary laboratory data 124 or infection type data 128 may be difficult to obtain in some hospitals or healthcare facilities. Thus, if the first LoS estimation model 108a is only slightly less accurate than the second or third LoS estimation models 108b, c, the first LoS estimation model 108a may be preferred.

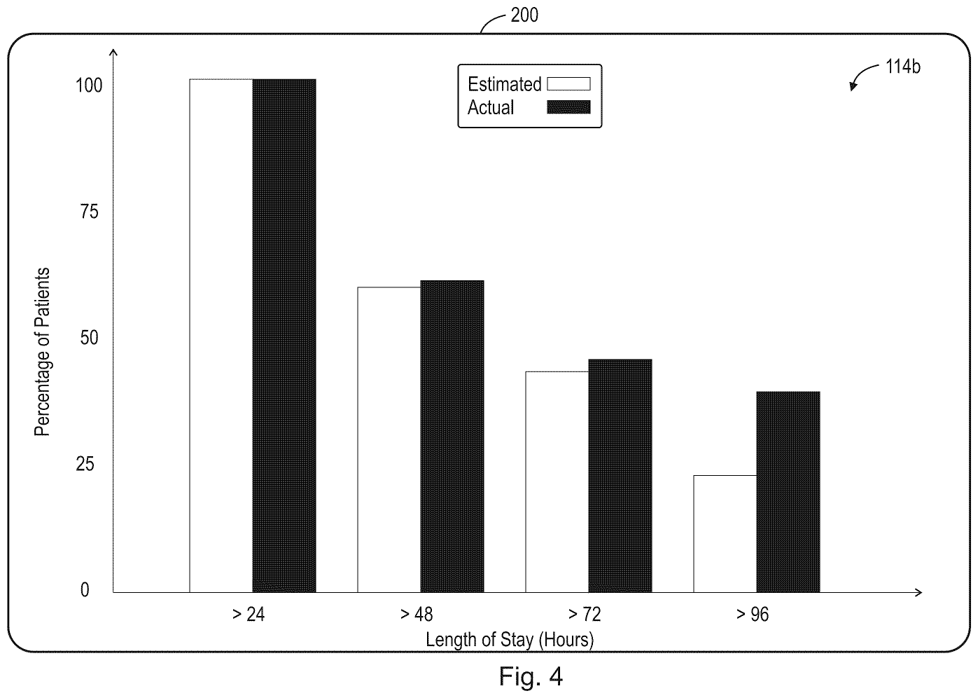

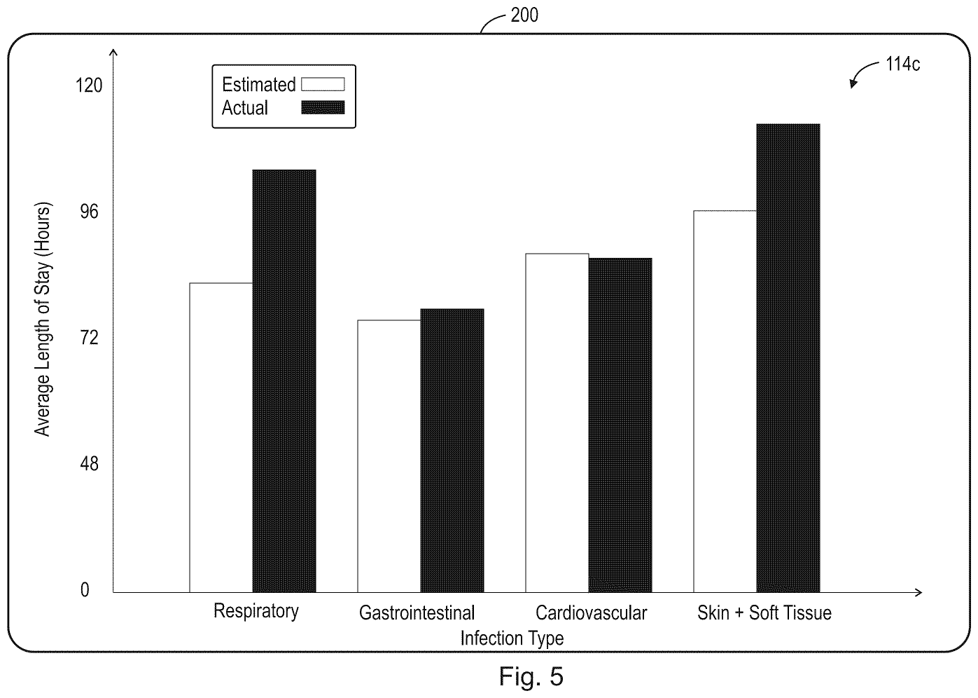

Once trained, the LoS estimation model 108 generates LoS estimations 110 for each filtered patient encounter record 102. These LoS estimations 110, along with the patient encounter records 102, are provided to a metric analyzer 112. Broadly, the metric analyzer 112 generates one or more evaluation metrics 114 by comparing the LoS estimations 110 to the patient LoS data 104. In one example, a first evaluation metric 114a compares LoS estimations 110 to patient LoS data 106 across age categories (see FIG. 3). In another example, a second evaluation metric 114b breaks down the lengths of stay into percentage of total patients (see FIG. 4). In an even further example, a third evaluation metric 114c compares LoS estimations 110 to patient LoS data 106 across infection type categories (see FIG. 5). In further examples, the metric analyzer 112 may generate additional types of evaluation metrics 114 according to the present application.

The system 10 further includes a user interface 200. The user interface 200 may be a display screen configured to show certain aspects of the system 10 to staff members of the hospital or healthcare facility. In some examples, the user interface 200 may be embedded into a control center or control station of the hospital or healthcare facility. For example, the user interface 200 may receive, via wired or wireless transmission, and subsequently display, the evaluation metrics 114 from the metric analyzer 112.

FIG. 3 illustrates an example of the user interface 200 of FIG. 2. The user interface 200 of FIG. 3 may be a display screen, such as a touch screen. The user interface 200 may be embedded in an existing control station or console, or it may be a part of a portable device, such as a tablet computer or mobile device. Generally, the user interface 200 is configured to display the aforementioned evaluation metrics 114 to the staff members of the hospital or healthcare facility so that policies and procedures may be updated to improve performance. In the example of FIG. 3 showing a first evaluation metric 114a, the average estimated patient length of stay is compared to the average actual length of stay for infection patients in four different age groups: 18-34, 35-49, 50-64, and 65 and over. As previously mentioned, patients under the age of 18 are excluded from the evaluation process. As shown in FIG. 3, the average actual length of stay for patients in the 18-34 and 35-49 age groups is slightly greater than the average estimated length of stay, possibly indicative of minor inefficiencies in infection treatment. Further, the average actual length of stay for patients in the 50-64 age group is slightly less than the average estimated length of stay, possibly indicative of effective and efficient treatment. Even further, the average actual length of stay of patients in the 65+ age group is significantly higher than the average estimated length of stay, possibly indicative of significant systemic issues with treating patients in this age group.

FIG. 4 illustrates a further example of the user interface 200 showing a second evaluation metric 114b. The second evaluation metric 114b of FIG. 4 shows the estimated and actual percentage of total patients having lengths of stay above certain thresholds. As can be seen, the estimated and actual percentage of patients having a length of stay of greater than 24 hours is 100%, as the system 10 only estimates lengths of stay for patients receiving inpatient treatment. Further, the actual percentage of patients having lengths of stay greater than 48 or 72 hours is slightly greater than the estimated percentage, possibly indicative of effective and efficient treatment. Even further, the actual percentage of patients having a length of stay greater than 96 hours is significantly greater than the estimated percentage, possibly indicative of significant systemic issues with treating patients with more severe infections.

FIG. 5 illustrates a further example of the user interface 200 showing a third evaluation metric 114c. The third evaluation metric 114c of FIG. 5 compares the average estimated length of stay to the average actual length of stay for patients diagnosed with four types of infections: respiratory, gastrointestinal, cardiovascular, and skin and soft tissue. As shown in FIG. 5, the average actual length of stay for patients with gastrointestinal infections is slightly greater than the average estimated length of stay, possibly indicative of minor inefficiencies in gastrointestinal infection treatment. Further, the average actual length of stay for patients with cardiovascular infections is slightly less than the average estimated length of stay, possibly indicative of effective and efficient treatment of cardiovascular infection. Even further, the average actual length of stay of patients with respiratory and skin and soft tissue infections is significantly higher than the average estimated length of stay, possibly indicative of significant systemic issues with treating patients with these infections.

FIG. 6 schematically illustrates the controller 100 previously depicted in FIG. 1. The controller 100 includes the processor 125, the memory 175, and the transceiver 185. The memory 175 is configured to store the patient encounter records 102, the LoS estimations 110, the evaluation metrics 114, the contemporary EMR 118, the historical encounter records 134, the historical EMR 156, the concordance index 158, and the regularization parameters 164. The patient encounter records 102 include patient attribute data 104 and patient LoS data 106. The historical encounter records 134 include historical attribute data 136 and historical recovery data 138. The historical recovery data 138 includes right-censored recovery data 140. The historical encounter records 134 may be divided into a plurality of (such as five) folds 160. The processor 125 is configured to execute the LoS execution model 108, the metric analyzer 112, the feature extractor 116, the data filter 166, and the data labeler 168.

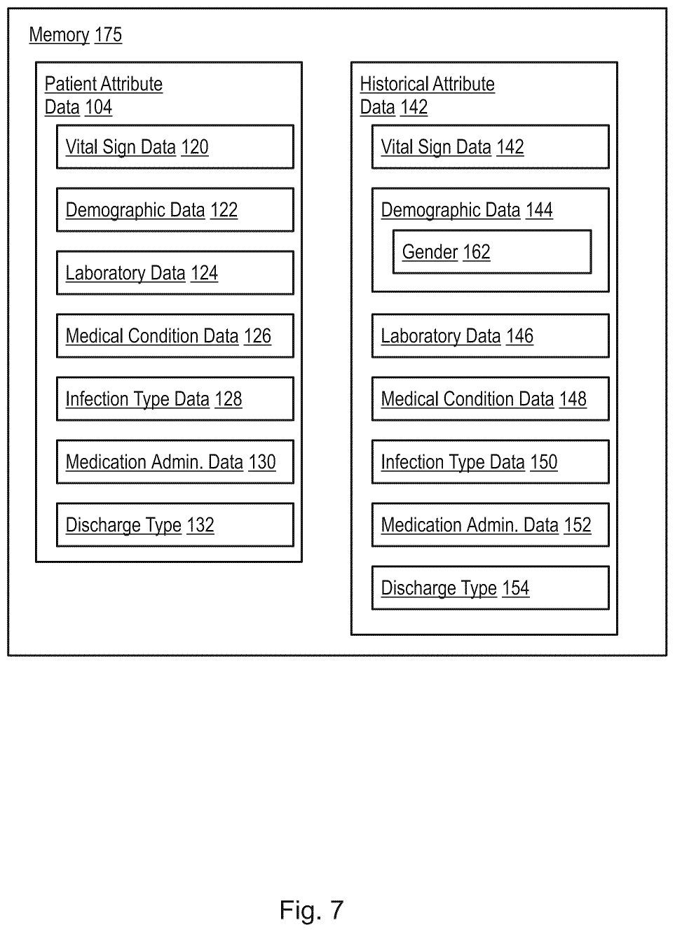

FIG. 7 schematically illustrates further aspects of the memory 175 of FIG. 6. The patient attribute data 104 includes vital sign data 120, patient demographic data 122, laboratory data 124, medical condition data 126, infection type data 128, medication administration data 130, and discharge type 132. The historical attribute data 142 includes historical vital sign data 142, historical demographic data 144, historical laboratory data 146, historical medical condition data 148, historical infection type data 150, historical medication administration data 152, and historical discharge type 154. The historical patient demographic data 144 includes gender 162.

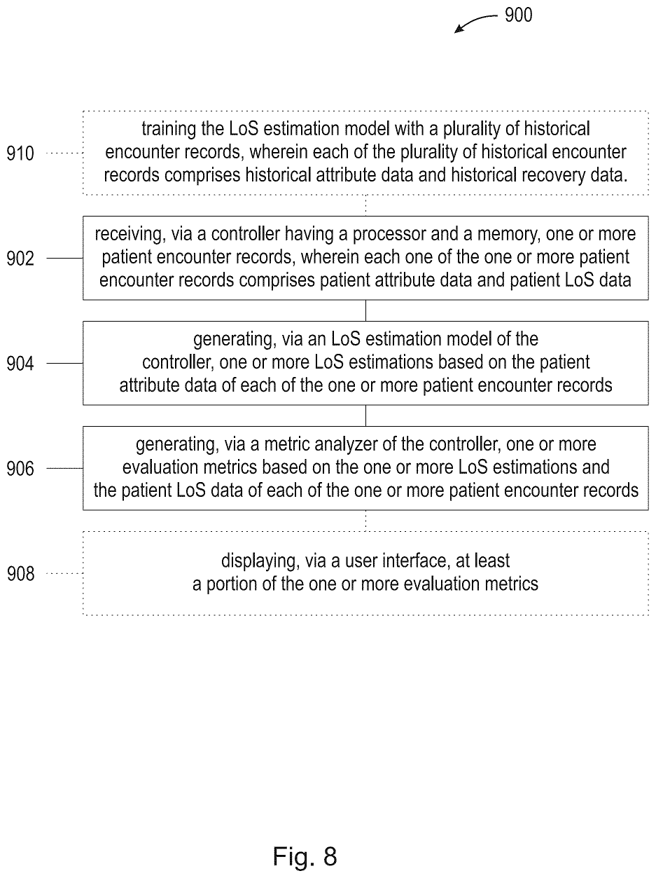

FIG. 8 is a flow chart of a method 900 for infection treatment evaluation of a hospital or healthcare facility. Referring to FIGS. 1-8, the method 900 includes, in step 902, receiving, via a controller 100 having a processor 125 and a memory 175, one or more patient encounter records 102. Each one of the one or more patient encounter records 102 comprises patient attribute data 104 and patient LoS data 106.

The method 900 further includes, in step 904, generating, via an LoS estimation model 108 of the controller 100, one or more LoS estimations 110 based on the patient attribute data 104 of each of the one or more patient encounter records 102.

The method 900 further includes, in step 906, generating, via a metric analyzer 112 of the controller 100, one or more evaluation metrics 114 based on the one or more LOS estimations 110 and the patient LoS data 106 of each of the one or more patient encounter records 102.

According to an example, the method 900 further includes, in optional step 908, displaying, via a user interface 200, at least a portion of the one or more evaluation metrics 114.

According to an example, the method 900 further includes, in optional step 910, training the LoS estimation model 108 with a plurality of historical encounter records 134, wherein each of the plurality of historical encounter records 134 comprises historical attribute data 136 and historical recovery data 138.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software, or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A system for infection treatment evaluation comprising a controller having a processor and a memory, wherein the controller is configured to:
receive one or more patient encounter records, wherein each of the one or more patient encounter records comprises patient attribute data and patient length of stay (LoS) data;
generate, via an LoS estimation model, one or more LoS estimations based on the patient attribute data of each of the one or more patient encounter records; and
generate, via a metric analyzer, one or more evaluation metrics based on the one or more LoS estimations and the patient LoS data of each of the one or more patient encounter records.

2. The system of claim 1, further comprising a user interface configured to display at least a portion of the one or more evaluation metrics.

3. The system of claim 1, wherein the controller is further configured to extract, via a feature extractor, at least a portion of the one or more patient encounter records from contemporary electronic medical records (EMR).

4. The system of claim 1, wherein the patient attribute data comprises vital sign data, patient demographic data, laboratory data, medical condition data, infection type data, medication administration data, and/or discharge type.

5. The system of claim 1, wherein the controller is further configured to train the LoS estimation model with a plurality of historical encounter records, wherein each of the plurality of historical encounter records comprises historical attribute data and historical recovery data.

6. The system of claim 5, wherein the historical recovery data comprises right-censored recovery data corresponding to patient discharge prior to recovery.

7. The system of claim 5, wherein the historical attribute data comprises historical vital sign data, historical demographic data, historical laboratory data, historical medical condition data, historical infection type data, historical medication administration data, and/or historical discharge type.

8. The system of claim 5, wherein the controller is further configured to extract, via a feature extractor, at least a portion of the plurality of historical encounter records from historical electronic medical records (EMR).

9. The system of claim 5, wherein the controller is further configured to generate, via the plurality of historical encounter records, a concordance index value of the LoS estimation model.

10. The system of claim 9, wherein the controller is further configured to divide the plurality of historical encounter records into five folds.

11. The system of claim 10, wherein the plurality of historical encounter records is divided in the five folds based on historical gender and historical discharge type.

12. The system of claim 9, wherein the controller is further configured to adjust one or more regularization parameters of the LoS estimation model based on the concordance index value.

13. A method for infection treatment evaluation, comprising:
receiving, via a controller having a processor and a memory, one or more patient encounter records, wherein each one of the one or more patient encounter records comprises patient attribute data and patient length of stay (LoS) data;
generating, via an LoS estimation model of the controller, one or more LoS estimations based on the patient attribute data of each of the one or more patient encounter records; and
generating, via a metric analyzer of the controller, one or more evaluation metrics based on the one or more LOS estimations and the patient LoS data of each of the one or more patient encounter records.

14. The method of claim 13, further comprising displaying, via a user interface, at least a portion of the one or more evaluation metrics.

15. The method of claim 13, further comprising training the LoS estimation model with a plurality of historical encounter records, wherein each of the plurality of historical encounter records comprises historical attribute data and historical recovery data.
